(19) United States
(12) Patent Application Publication
(10) Pub. No.: US 2019/0223712 A1
(43) Pub. Date: Jul. 25, 2019

(54) EYELID OPENER AND EYELID OPENING AND SENSING DEVICE

(71) Applicant: UBIQUITY BIOMEDICAL CORPORATION, Taipei City (TW)

(72) Inventors: HORNG-JI LAI, TAIPEI CITY (TW); LI-PING YU, TAIPEI CITY (TW); HSIANG-CHING LEE, TAIPEI CITY (TW); TE-LUN MA, TAIPEI CITY (TW); CHANG-YUAN FAN, TAIPEI CITY (TW); CHEN-HAO KAN, TAIPEI CITY (TW)

(21) Appl. No.: 15/878,770

(22) Filed: Jan. 24, 2018

(51) Int. Cl. A61B 3/10; A61B 5/01

(52) U.S. Cl. CPC A61B 3/10; A61B 17/0231; A61B 5/01

(57) ABSTRACT

An eyelid opener and an eyelid opening and sensing device are provided. The eyelid opener has a housing, two eyelid pushers and one eye positioning unit. The eye positioning unit abuts the surrounding of the eye socket for positioning (e.g. one side of the nasal bridge), and then the sensing process (e.g. eye pressure, eye temperature) of an eyeball sensor is started, ensuring the inspected region remains the same for each measurement. By contacting the user's face, the eyelid opener can be operated by the user independently. The positioning process ensures the eyeball sensor and the eyeball are placed at a fixed distance to prevent the eyeball sensor from physical contacting or hurting the eyeball and to facilitate mounting the eyeball sensor near the eyeball. The two eyelid pushers can avoid unconscious blinking during the sensing process.

### BACKGROUND OF THE INVENTION

### 1. Field of the Invention

The present invention relates to an eyelid opener and a combination of an eyelid opener and a sensor, especially to an assembly of the eyelid opener and an eye temperature sensing device.

### 2. Description of the Prior Arts

For diagnosis of eye diseases, conventional means rarely include the measurement of the eye temperature. However, symptoms such as dry eye syndrome (DES), cataract and eyestrain could be diagnosed through the measurement of the eye temperature. As the eye temperature is not commonly measured in the aforementioned conventional means, there's no customized equipment for eye temperature measurement. Though users may take a conventional non-contact forehead thermometer as a temporary solution, the non-contact forehead thermometer, when used as an eye temperature measuring device, has several defects.

First, the non-contact forehead thermometer is not directly in contact with the user's eyeball or the surrounding of the eye socket during the sensing process. The non-contact forehead thermometer is suspending in the air and cannot be accurately aligned with the eyeball, which means that the non-contact forehead thermometer may be in an unstable condition during the sensing process. The non-contact feature may cause the deviation of the measured area from one measurement to another. Therefore, the void between the non-contact forehead thermometer and the skin of the user might affect the accuracy of the measurement.

Second, the inaccuracy due to the non-contact with the user's skin might be resolved with the assistance from another person. In other words, the eyeball temperature measurement by non-contact forehead thermometer is not user-friendly for the user to use it independently.

Third, when using the non-contact thermometer, the distance between the non-contact thermometer and the surface of the skin is preferably at least 3 to 4 centimeters. To accurately measure the temperature of the eyeball, the non-contact forehead thermometer needs to be closer to the eyeball. However, due to the non-contact with the user's skin, shortening the distance between the non-contact thermometer and the surface of the skin raises the risk of contacting the eyeball and causing discomfort of the user.

Fourth, the user may blink during the measurement, which also affects the accuracy of the result. Moreover, the non-contact thermometer is not easy to be orientated in the correct position, and thus it may take longer time to adjust to the right position and to obtain the data accurately, which also increases the chance of the user's unconscious blinking, further affecting the accuracy of the result.

The defects as shown above could also occur in any measurement that requires the accurate positioning and the non-blinking eyes such as the measurement of the eye pressure. Therefore, the conventional means of collecting the physiological parameters about a user's eye needs to be improved.

To overcome the shortcomings, the present invention provides an eyelid opener and an eyelid opening and sensing device to mitigate or obviate the aforementioned problems.

### SUMMARY OF THE INVENTION

The main objective of the present invention is to provide an eyelid opener and an eyelid opening and sensing device that can be positioned easily and accurately, meanwhile holding the eyelids to remain open to facilitate the ocular examination.

The eyelid opener has a housing, an upper eyelid pusher, a lower eyelid pusher, an upper pushing mechanism, a lower pushing mechanism, and an eye positioning unit. The housing has an inner space and an opening, and the opening communicates with the inner space. The upper eyelid pusher is disposed in the housing, protrudes out of the opening of the housing, and is for pushing the user's upper eyelid. The lower eyelid pusher is disposed in the housing, protrudes out of the opening of the housing, and is for pushing the user's lower eyelid. The upper pushing mechanism is connected to the upper eyelid pusher and is capable of moving the upper eyelid pusher upwardly so as to push the user's upper eyelid upwardly. The lower pushing mechanism is connected to the lower eyelid pusher and is capable of making the lower eyelid pusher push the user's lower eyelid downwardly. The eye positioning unit is connected to the housing and is for annularly contacting a surrounding of the user's eye socket.

The eyelid opening and sensing device comprises the eyelid opener as aforementioned and an eyeball sensor. The eyeball sensor is mounted securely in the housing of the eyelid opener and is for sensing physiological parameters of the user's eyeball via the opening of the housing.

Given the foregoing structure of the eyelid opener and eyelid opening and sensing device, by further comprising the eye positioning unit, the eyelid opener is capable of contacting the surrounding of the user's eye socket (e.g. a side of the nasal bridge) before the sensing device is started, thereby ensuring that the measurement is carried out in the same position each time, avoiding the inaccuracy of the physiological parameters caused by inaccurate positioning.

Moreover, positioning the eyelid opener by an eye positioning unit enables the user to carry out the measurement independently without the assistance from others. Even if the user positions the eyeball sensor near the opening of the housing to shorten the distance between the eyeball sensor and the eyeball, the eye positioning unit can still maintain a fixed distance between the eyeball sensor and the eyeball, preventing the eyeball sensor from contacting the eyeball or even hurting the eyeball.

By shortening the distance between the eyeball sensor and the eyeball, inaccuracy caused by the eyelids is reduced by means of precisely sensing the surrounding of the pupil of the eye in a short distance.

Lastly, the upper eyelid pusher and the lower eyelid pusher are capable of holding open the upper eyelid and the lower eyelid respectively, thereby preventing the user from blinking and also ensuring accuracy of the subsequent sensing process by the eyeball sensor.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of an eyelid opener in accordance with the present invention;

FIG. 2 is another perspective view of the eyelid opener in FIG. 1;

FIG. 3 is an exploded perspective view of the eyelid opener in FIG. 1;

FIG. 4 is an exploded perspective view of the upper pushing mechanism and the lower pushing mechanism in FIG. 1;

FIG. 5 is an exploded perspective view of the nasal bridge assembly and the nasal bridge positioning unit in FIG. 1;

FIG. 6 is a perspective view of the abutting part with the adjusting unit in FIG. 1;

FIG. 7 is an operational view of the nasal bridge positioning unit in FIG.

1;

FIG. 8 is an operational view of the eyelid opener in FIG. 1 relative to the user's eye socket and its surrounding area;

FIG. 9 is an operational view of the nasal bridge positioning unit in FIG. 1;

FIG. 10 is an operational view of the eyelid opener in FIG. 1;

FIG. 11 is another operational view of the eyelid opener in FIG. 1;

FIG. 12 is still another operational view of the eyelid opener in FIG. 1;

FIG. 13 is a cross sectional view of another embodiment of the lower pushing mechanism and the lower eyelid pusher of the eyelid opener in accordance with the present invention;

FIG. 14 is a perspective view of the second embodiment of the eyelid opener in accordance with the present invention;

FIG. 15 is an exploded view of the second embodiment of the eyelid opener in FIG. 14;

FIG. 16 is an operational view of the second embodiment of the eyelid opener in FIG. 14;

FIG. 17 is another operational view of the second embodiment of the eyelid opener in FIG. 14; and

FIG. 18 is a schematic view of an eyeball sensor, a triggering device, and a recording device of the eyelid opener in accordance with the present invention.

### DETAILED DESCRIPTION OF THE PREFERRED EMBODIMENTS

The present invention of an eyelid opening and sensing device comprises an eyelid opener, an eyeball sensor and a driving device. The eyelid opener is used to hold the user's upper eyelid and lower eyelid open. The eyeball sensor is disposed in the eyelid opener and is capable of extracting various physiological parameters from the user's eyeball. In the present embodiment, the eyeball sensor is, but not limited to, an eyeball temperature sensing device, as the eyeball sensor may also be, for example, an eye pressure sensing device.

The eyelid opener is used to open and hold the user's upper eyelid and lower eyelid. The present invention has two embodiments.

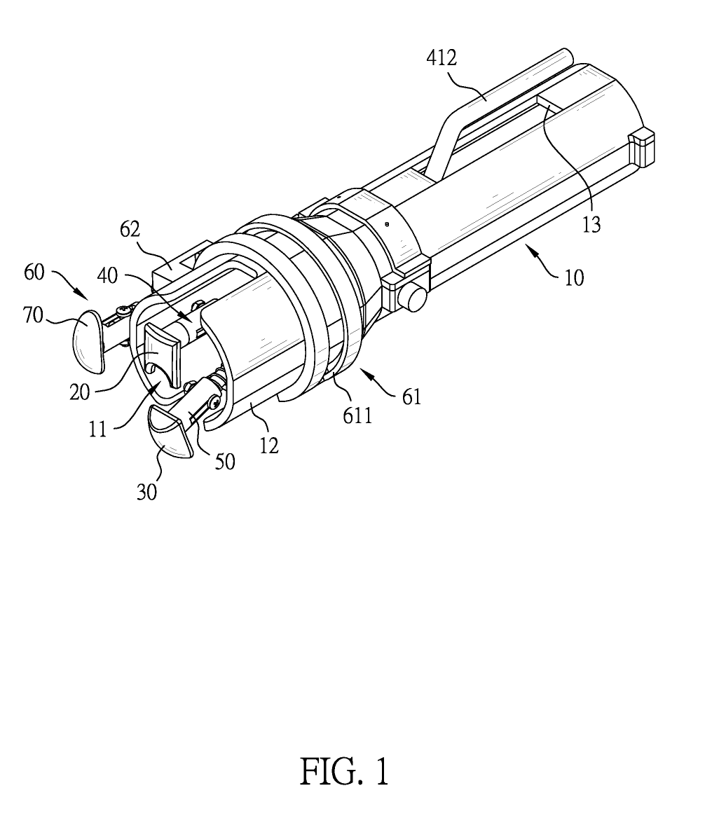

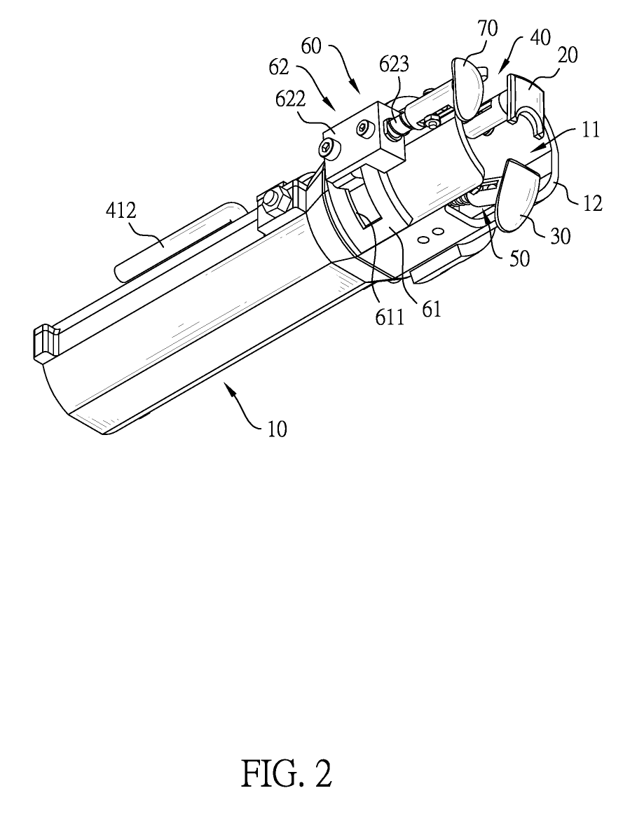

With reference to FIG. 1 to FIG. 3, the first embodiment of an eyelid opener in accordance with the present invention comprises a housing 10, an upper eyelid pusher 20, a lower eyelid pusher 30, an upper pushing mechanism 40, a lower pushing mechanism 50, a nasal bridge assembly 60 and an eye positioning unit. In the first embodiment, the eye positioning unit is a nasal bridge positioning unit 70.

The housing 10 has an inner space and an opening 11, and the inner space and the opening 11 are mutually communicating with each other. In the first embodiment, the housing 10 is, but not limited to, roughly in a round tube. In the first embodiment, a light shielding sleeve 12 is formed by a surrounding surface of the housing 10 which is adjacent to the opening 11. When in use, the light shielding sleeve 12 is not directly in contact with the user's skin, but the light shielding sleeve 12 can shield the light from the user's sight, thereby offering a stabilized sensing environment.

The eyeball sensor is disposed in the housing 10. In a preferred embodiment, the eyeball sensor may be mounted on the housing 10. But the eyeball sensor also may be mounted on other component of the eyelid opener.

The upper eyelid pusher 20 and the lower eyelid pusher 30 are both disposed in the housing 10 and both protrude from the opening 11 of the housing 10. The upper eyelid pusher 20 is for pushing and holding the user's upper eyelid, and the lower eyelid pusher 30 is for pushing and holding the user's lower eyelid.

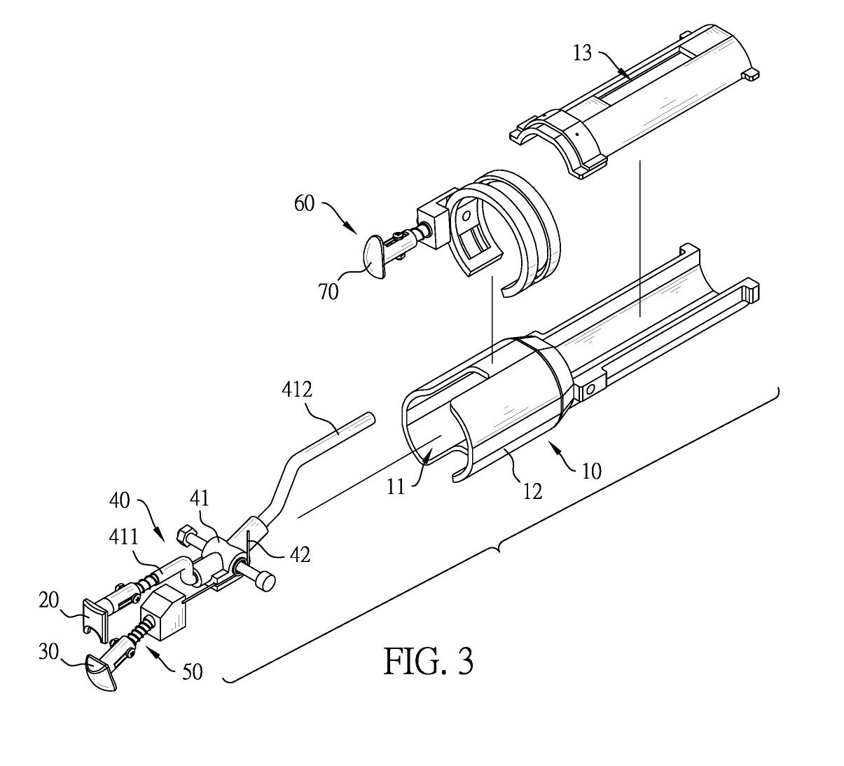

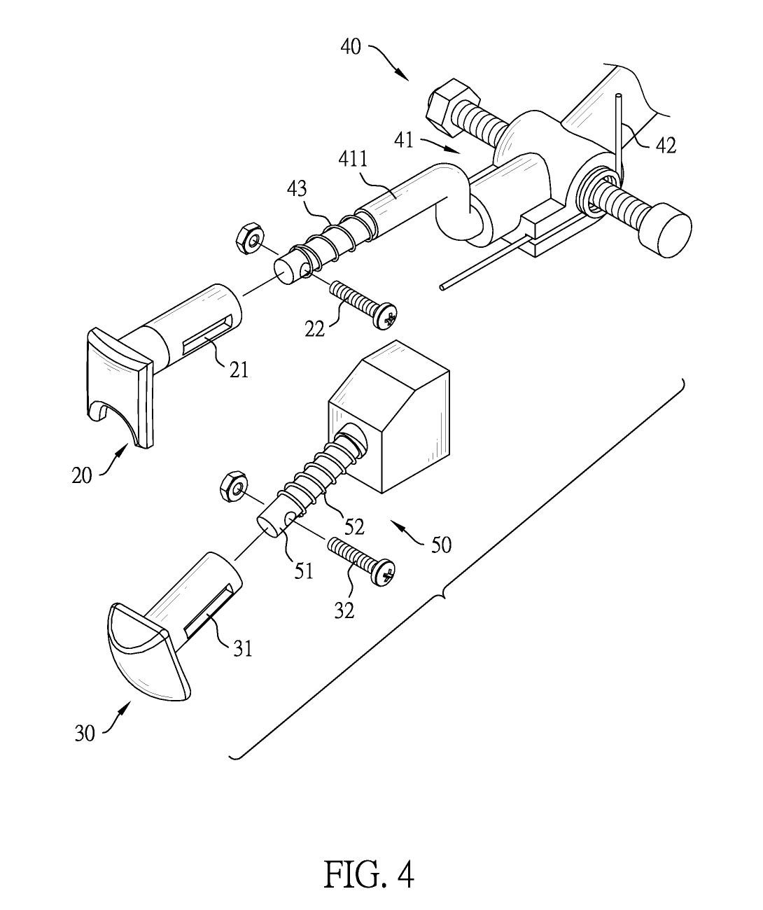

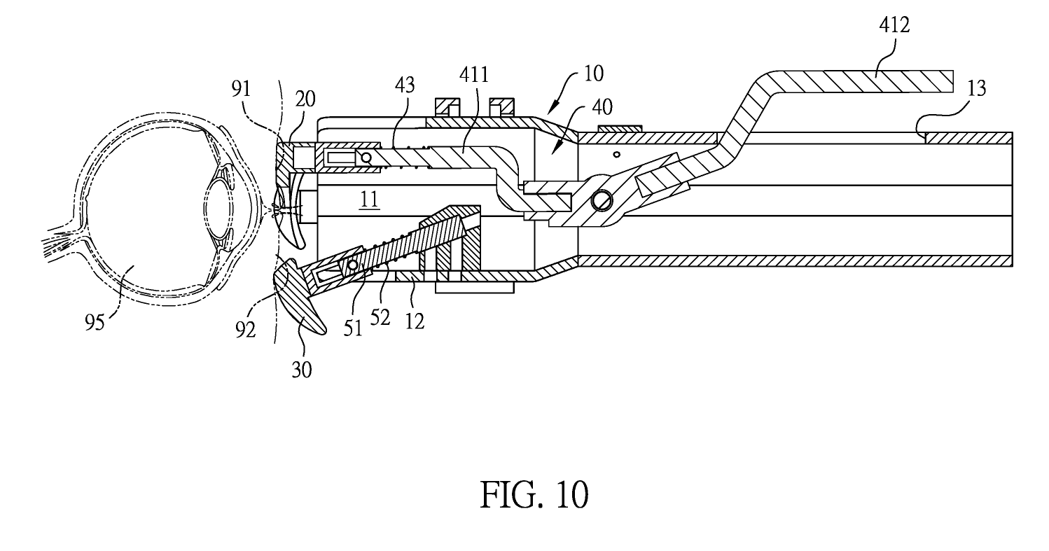

With reference to FIG. 3, FIG. 4 and FIG. 10, the upper pushing mechanism 40 is connected to the upper eyelid pusher 20. The upper pushing mechanism 40 is capable of moving the upper eyelid pusher 20 upwardly to push the user's upper eyelid 91 upwardly.

The lower pushing mechanism 50 is connected to the lower eyelid pusher 30. The lower pushing mechanism 50 is capable of making the lower eyelid pusher 30 push the user's lower eyelid 92 downwardly.

Specifically, in the first embodiment, the upper pushing mechanism 40 comprises a main body 41, a first resilient element 42 and a second resilient element 43.

The main body 41 is mounted in the housing 10 and is capable of pivoting up and down. Two opposite ends of the main body 41 are a connecting end 411 and an operating end 412 respectively. The connecting end 411 extends toward the opening 11 of the housing 10 and connects to the upper eyelid pusher 20. The operating end 412 protrudes from the housing 10, and the operating end 412 is pressable to operate by the user. In the first embodiment, a perforation 13 is formed on a surface of the housing 10, and the operating end 412 protrudes from the housing 10 through the perforation 13, but it is not limited thereto, as the operating end 412 can also protrude from an opposite end relative to the opening 11, as long as it can be easily pressed by the user.

The first resilient element 42 is connected to the main body 41 and the housing 10. The first resilient element 42 is capable of pushing the main body 41 toward a direction that is opposite to the direction in which the main body 41 pushes the upper eyelid 91. In other words, the first resilient element 42 constantly pushes the connecting end 411 of the main body 41 to pivot downwardly. To hold the upper eyelid 91 open, press the operating end 412 of the main body 41. In the first embodiment, the first resilient element 42 is, but not limited to, a torsion spring.

The second resilient element 43 is mounted on the connecting end 411 of the main body 41. The second resilient element 43 pushes the upper eyelid pusher 20 toward the opening 11 of the housing 10. Therefore, when the upper eyelid pusher 20 abuts against the user's upper eyelid 91, the upper eyelid pusher 20 can slightly move inwardly toward the inner space of the housing 10. In the first embodiment, the second resilient element 43 is, but not limited to, a compression spring.

In the first embodiment, the upper eyelid pusher 20 is mounted around the connecting end 411 of the main body 41. The upper eyelid pusher 20 has an elongated hole 21 and a first limiting unit 22. The elongated hole 21 is formed on the upper eyelid pusher 20. The first limiting unit 22 is formed through the elongated hole 21 and is mounted securely on the connecting end 411 of the main body 41. Therefore the first limiting unit 22 limits the upper eyelid pusher 20 to be only capable of moving straightly relative to the connecting end 411. In the first embodiment, the first limiting unit 22 comprises a bolt and a screw, but it is not limited thereto.

In the first embodiment, the lower pushing mechanism 50 comprises a lower rod 51 and a third resilient element 52.

The lower rod 51 extends obliquely downwardly toward the opening 11 of the housing 10. The lower rod 51 is connected to the lower eyelid pusher 30.

The third resilient element 52 is mounted on the lower rod 51. The third resilient element 52 pushes the lower eyelid pusher 30 toward the opening 11 of the housing 10. Therefore, when the lower eyelid pusher 30 abuts against the user's lower eyelid 92, the lower eyelid pusher 30 can slightly move inwardly toward the inner space of the housing 10. In the first embodiment, the third resilient element 52 is, but not limited to, a compression spring.

In the first embodiment, the lower eyelid pusher 30 is mounted around the lower rod 51. The lower eyelid pusher 30 has an elongated hole 31 and a second limiting unit 32. The elongated hole 31 is formed on the lower eyelid pusher 30. The second limiting unit 32 protrudes from the elongated hole 31 and is mounted securely on the lower rod 51. Therefore the second limiting unit 32 limits the lower eyelid pusher 30 to be only capable of moving straightly relative to the lower rod 51. In the first embodiment, the second limiting unit 32 comprises a bolt and a screw, but it is not limited thereto.

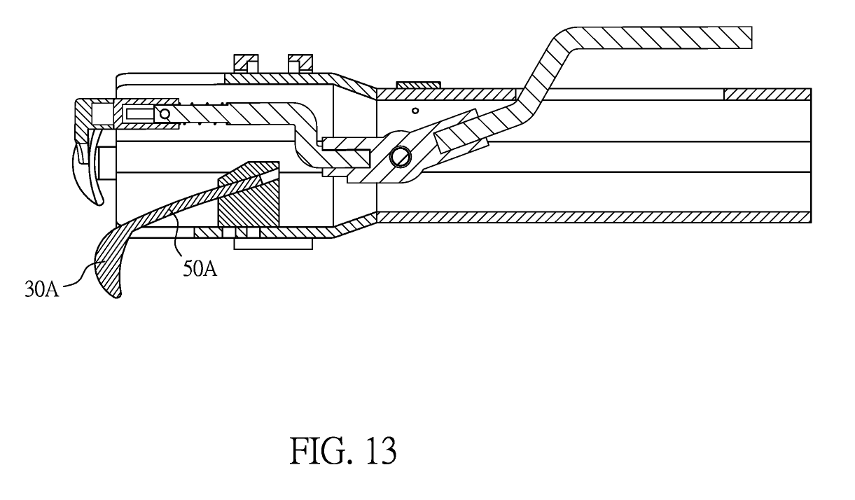

With reference to FIG. 13, in other embodiments, the lower pushing mechanism 50A may be made of elastic materials. The lower eyelid pusher 30A may be connected to or integrated with the lower pushing mechanism 50A. Preferably, the lower pushing mechanism 50A and the lower eyelid pusher 30A may jointly form in a spoon-like shape. Therefore when the user pushes the eyelid opener toward his/her face, the deformation of the lower pushing mechanism 50A can cause the lower eyelid pusher 30A to push the user's lower eyelid 92 downwardly, thereby achieving the same result as the first embodiment.

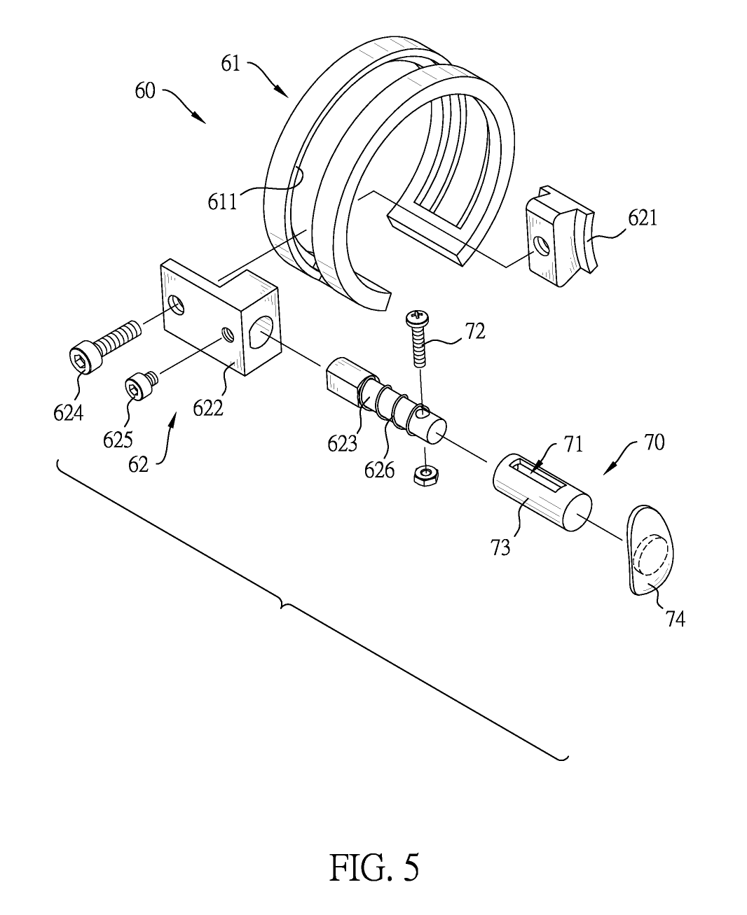

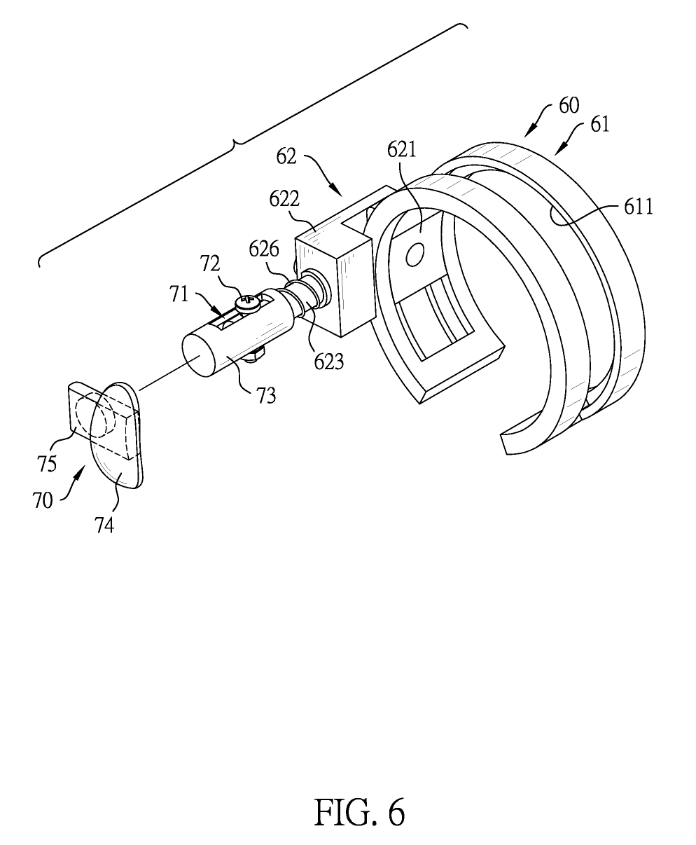

With reference to FIG. 5 to FIG. 7 and FIG. 10, the nasal bridge assembly 60 comprises a nasal bridge track 61 and a nasal bridge abutting base 62.

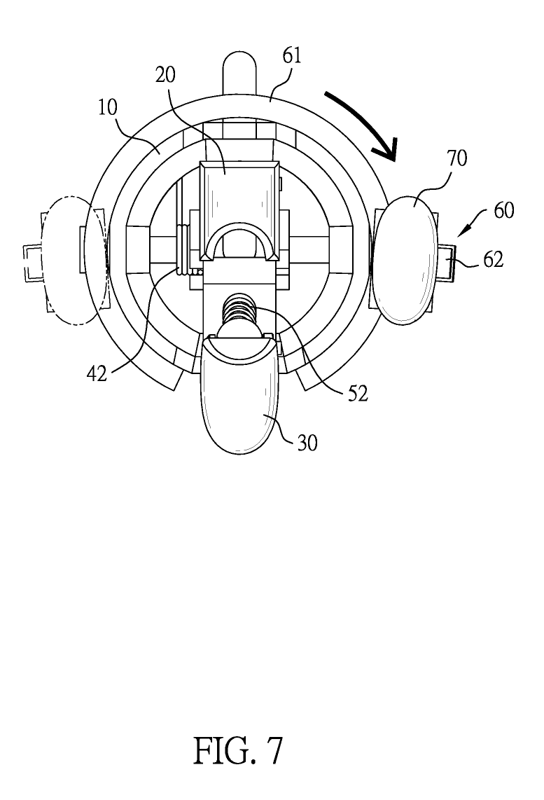

The nasal bridge track 61 is mounted on an outer surface of the housing 10, and extends annularly to the two horizontally opposite sides of the housing 10. The nasal bridge abutting base 62 is mounted on the nasal bridge track 61 and is capable of moving along the nasal bridge track 61. The nasal bridge abutting base 62 can move to the two horizontally opposite sides of the housing 10 through the nasal bridge track 61 respectively. Therefore, the nasal bridge positioning unit 70, which is mounted on the nasal bridge abutting base 62, can also move to the two horizontally opposite sides of the housing 10 respectively, thereby making the eyelid opener applicable to either the right eye or the left eye (as shown in FIG. 7).

In the first embodiment, a track hole 611 is formed on the nasal bridge track 61, and the track hole 611 annularly extends to the two horizontally opposite sides of the housing 10. The nasal bridge abutting base 62 comprises an inner part 621, an outer part 622, an installing rod 623, two screws 624, 625, and a fourth resilient element 626.

The inner part 621 is disposed on an inner side of the nasal bridge track 61, and the outer part 622 is disposed on an outer side of the nasal bridge track 61. The inner part 621 and the outer part 622 jointly clamp a surrounding edge of the track hole 611. An end of the installing rod 623 is mounted through the outer part 622. The inner part 621 and the outer part 622 are fastened by a screw 624, and the installing rod 623 is fastened in the outer part 622 by another screw 625.

The nasal bridge positioning unit 70 is mounted on the nasal bridge abutting base 62 and protrudes toward the opening 11 of the housing 10 for abutting against one side of the user's nasal bridge. Thus, the nasal bridge positioning unit 70 can position the eyelid opener by the means shown above.

Specifically, the two opposite ends of the installing rod 623 are mounted respectively by the outer part 622 and the nasal bridge positioning unit 70. An elongated hole 71 is formed on the nasal bridge positioning unit 70 and a third limiting unit 72 is mounted through the elongated hole 71 and is mounted securely on the installing rod 623. Therefore the third limiting unit 72 limits the nasal bridge positioning unit 70 to be only capable of moving straightly relative to the nasal bridge abutting base 62. In the first embodiment, the third limiting unit 72 comprises a bolt and a screw, but it is not limited thereto.

The fourth resilient element 626 is mounted on the installing rod 623. The fourth resilient element 626 pushes the nasal bridge positioning unit 70 toward the opening 11 of the housing 10. Therefore when the nasal bridge positioning unit 70 abuts against one side of the user's nasal bridge, the nasal bridge positioning unit 70 can slightly move toward the nasal bridge abutting base 62. In the first embodiment, the fourth resilient element 626 is, but not limited to, a compression spring.

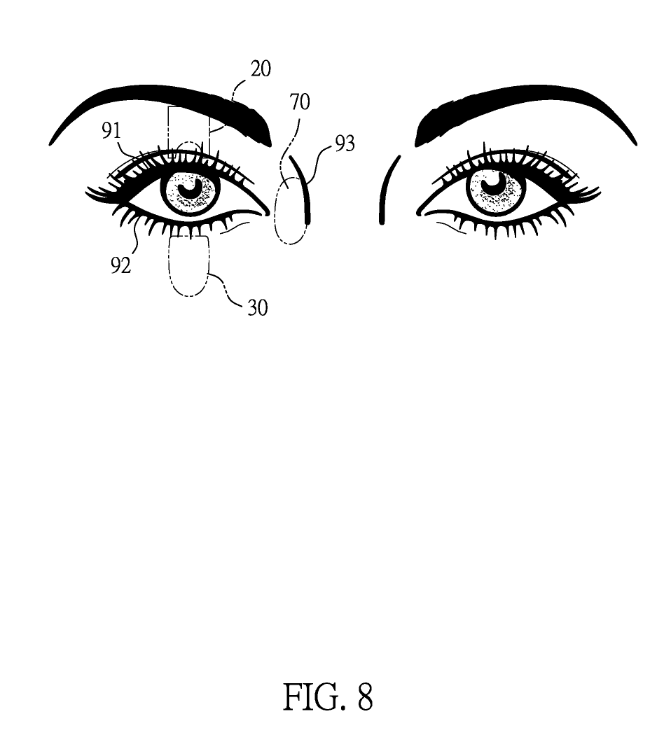

The nasal bridge positioning unit 70 protrudes from the opening 11 of the housing 10 farther than the lower eyelid pusher 30 (e.g. viewed from the side view as shown in FIG. 8). Meanwhile, the nasal bridge positioning unit 70 protrudes from the opening 11 of the housing 10 farther than the upper eyelid pusher 20. In addition, the lower eyelid pusher 30 protrudes from the opening 11 of the housing 10 farther than the upper eyelid pusher 20. In other words, when the user moves the eyelid opener toward his or her face, the nasal bridge positioning unit 70 will be the first element to contact the user's face (more precisely, the side of the nasal bridge). Then the lower eyelid pusher 30 abuts the face (lower eyelid 92), and the last is the upper eyelid pusher 20 abutting the face (upper eyelid 91), but it is not limited thereto. It may also be that the nasal bridge positioning unit 70, the lower eyelid pusher 30 and the upper eyelid pusher 20 protrude for an equal distance from the opening 11 of the housing 10, which means the nasal bridge positioning unit 70, the lower eyelid pusher 30 and the upper eyelid pusher 20 will contact the user's face at the same time.

Besides, in the first embodiment, the nasal bridge positioning unit 70 comprises an assembling base 73 and an abutting part 74. The assembling base 73 is connected to the nasal bridge abutting base 62, and the abutting part 74 is detachably mounted on the assembling base 73. The abutting part 74 is the element to abut against the side of the user's nasal bridge, and is available in different sizes and shapes for the user to choose and apply. When adjusting the distance in a larger scale, an abutting part 74 with an adjusting unit 75 is applicable. An end of the adjusting unit 75 is detachably mounted on the assembling base 73, and the abutting part 74 is mounted on the other end of the adjusting unit 75. Therefore, the distance between the abutting part 74 and the eyelid pushers 20, 30 is shortened.

Meanwhile, because the nasal bridge abutting base 62 is movably mounted on the nasal bridge track 61 and is also capable of being fixed on any spot along the nasal bridge track 61, it allows the user to further adjust the position of the nasal bridge positioning unit 70.

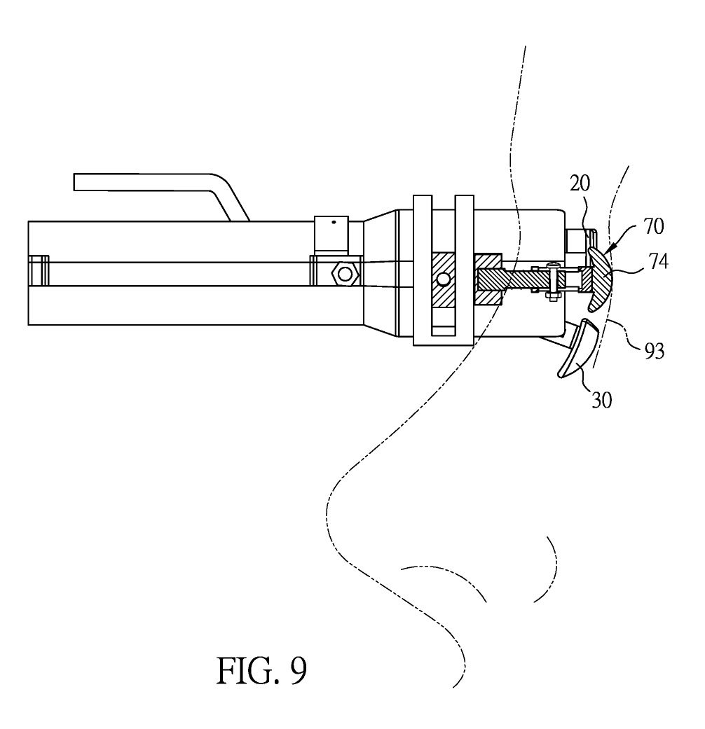

With reference to FIG. 8 to FIG. 11, when using the eyelid opener, the first thing to do is to abut the nasal bridge positioning unit 70 on one side 93 of the nasal bridge so as to position the eyelid opener. Then push the eyelid opener toward the face for the lower eyelid pusher 30 to abut against the lower eyelid 92. Keep pushing the eyelid opener until the upper eyelid pusher 20 abuts against the upper eyelid 91.

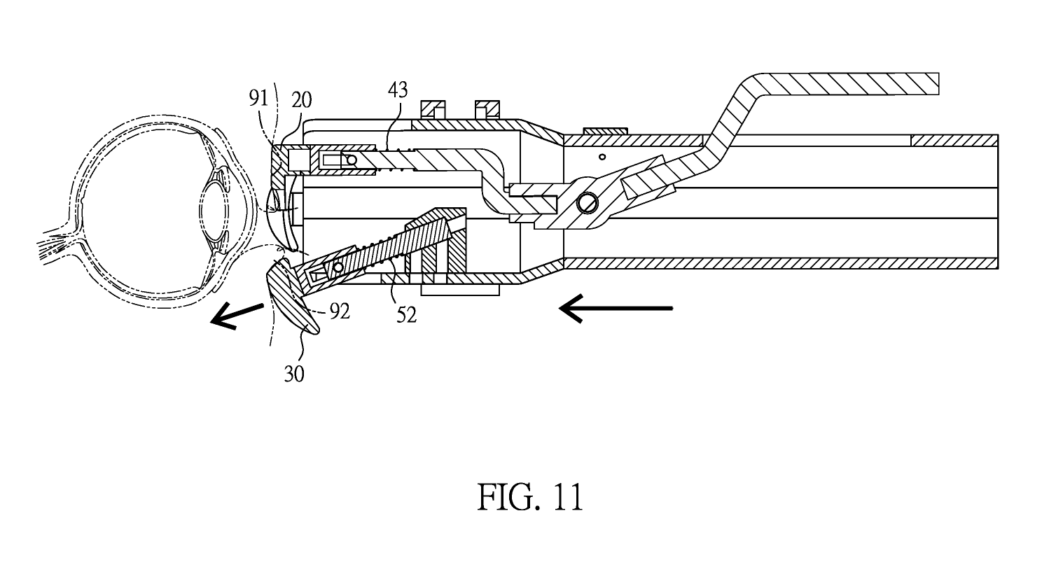

With reference to FIG. 10 and FIG. 11, after the lower eyelid pusher 30 has abutted against the lower eyelid 92 and before the upper eyelid pusher 20 abuts against the upper eyelid 91, the lower eyelid pusher 30 will push the lower eyelid 92 obliquely downwardly until the lower eyelid 92 is held open because the lower eyelid pusher 30 is mounted on the lower rod 51, which extends obliquely and downwardly.

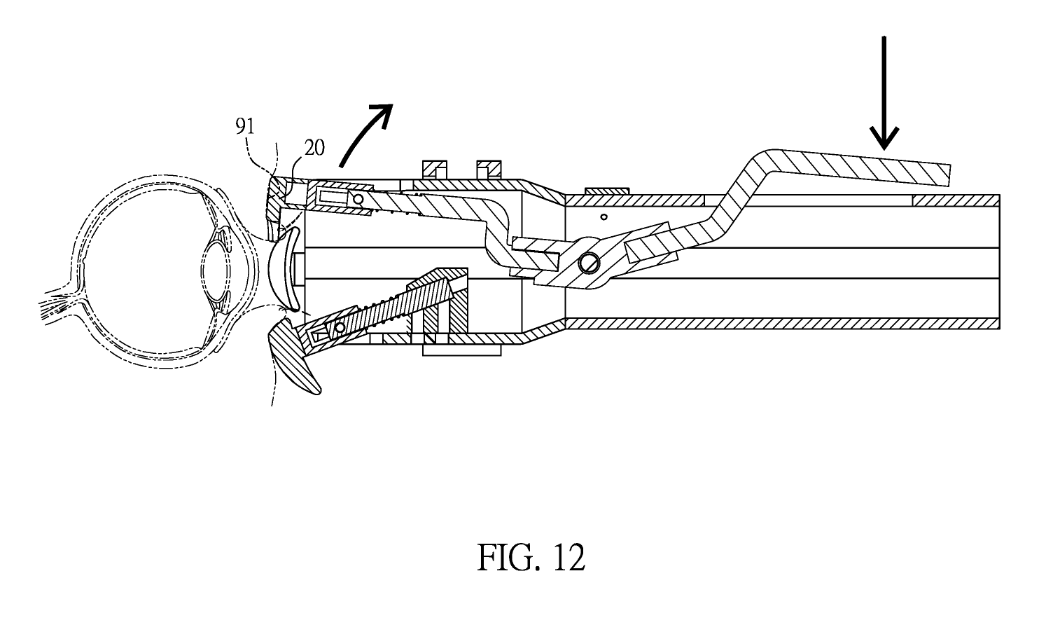

With reference to FIG. 11 and FIG. 12, after the upper eyelid pusher 20 abuts against the upper eyelid 91, press downwardly the operating end 412 of the main body 41 of the upper pushing mechanism 40 so as to pivot the main body 41 upwardly, thereby opening the upper eyelid 91 upwardly by the upper eyelid pusher 20 which is mounted on the connecting end 411 of the main body 41.

By abutting the nasal bridge positioning unit 70 against the side 93 of the user's nasal bridge, it can be ensured that the measuring position remain the same for each measurement, therefore the errors of the physiological parameters caused by positioning discrepancy can be avoided. Besides, the eyelid opener is physically contacted with the user's face during the measurement to be accurately positioned, and the operating method is fairly simple, so the user can operate the measurement process independently.

Moreover, even when the eyeball sensor is mounted adjacent to the opening 11 of the housing 10 to shorten the distance between the eyeball sensor and the eyeball 95, the distance between the eyeball 95 and the eyeball sensor can be kept in a safe distance by the nasal bridge positioning unit 70, therefore avoiding the physical contact of the eyeball sensor and the eyeball 95, which will cause harm to and discomfort of the user. On the other hand, by shortening the distance between the eyeball 95 and the eyeball sensor, the inaccuracy caused by the eyelids is lowered, increasing the accuracy of sensing the surroundings of the pupil. More precisely, in the first embodiment, the distance between the sensing region and the eyeball 95 can be shortened to a range from 1 cm to 2 cm.

Before the measurement, the upper eyelid 91 and the lower eyelid 92 will be held open beforehand by the eyelid opener, thereby avoiding blinking of eyes that interferes with the measurement.

By the means as shown above, the eyelid opener and the eyeball sensor are easy to use and are accurate with low sensing errors.

Furthermore, because the nasal bridge positioning unit 70, the lower eyelid pusher 30 and the upper eyelid pusher 20 can be operated separately, can be abutted against the user's face step by step, and each have their respective own resilient element, the shape, the position and the elasticity of each one of the elements can be adjusted according to the order of contact or the abutting site (e.g. the lower eyelid has a stronger tolerance, the upper eyelid has a weaker tolerance) to enhance the comfort in use.

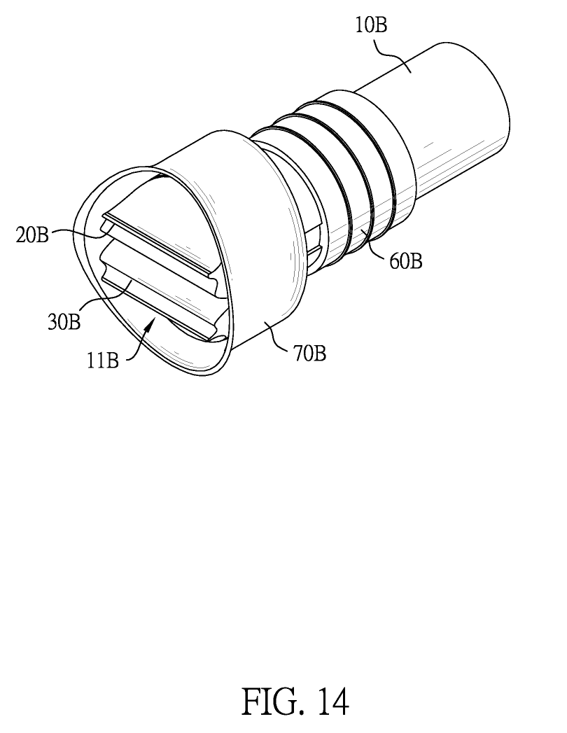

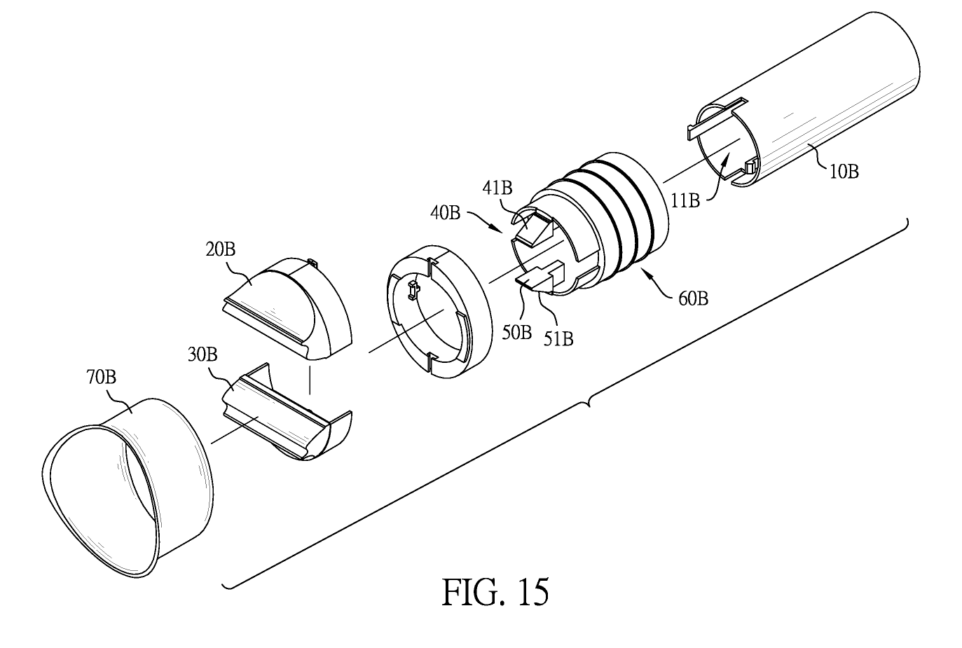

With reference to FIG. 14 to FIG. 16, the second embodiment is substantially the same with the first embodiment, except that in the first embodiment the upper eyelid pusher 20 is operated by press. In the second embodiment, the upper eyelid pusher 20B is operated by push. Specifically, the upper eyelid pusher 20B and the lower eyelid pusher 30B can be simultaneously moved by pushing.

More precisely, the second embodiment further comprises a pushing sleeve 60B mounted around the housing 10B. The pushing sleeve 60B is capable of moving toward the opening 11B of the housing 10B and selectively abuts against the upper pushing mechanism 40B and the lower pushing mechanism 50B. By pushing the pushing sleeve 60B, the upper eyelid pusher 20B will then push the user's upper eyelid 91B upwardly, and the lower eyelid pusher 30B will then push the user's lower eyelid 92B downwardly.

In a preferred embodiment, the pushing sleeve 60B is detachably mounted around the housing 10B, and thus the pushing sleeve 60B can be replaced.

Furthermore, in the second embodiment, the upper eyelid pusher 20B and the lower eyelid pusher 30B are both mounted in the housing 10B and are capable of moving upwardly and downwardly in the housing 10B respectively. The upper pushing mechanism 40B and the lower pushing mechanism 50B are both mounted securely on an end of the pushing sleeve 60B, which faces the opening 11B of the housing 10B. The upper pushing mechanism 40B and the lower pushing mechanism 50B are arranged apart from each other vertically. The upper pushing mechanism 40B has a first inclined surface 41B, the first inclined surface 41B faces upwardly and selectively abuts against the upper eyelid pusher 20B. The lower pushing mechanism 50B has a second inclined surface 51B, the second inclined surface 51B faces downwardly and is selectively abuts against the lower eyelid pusher 30B.

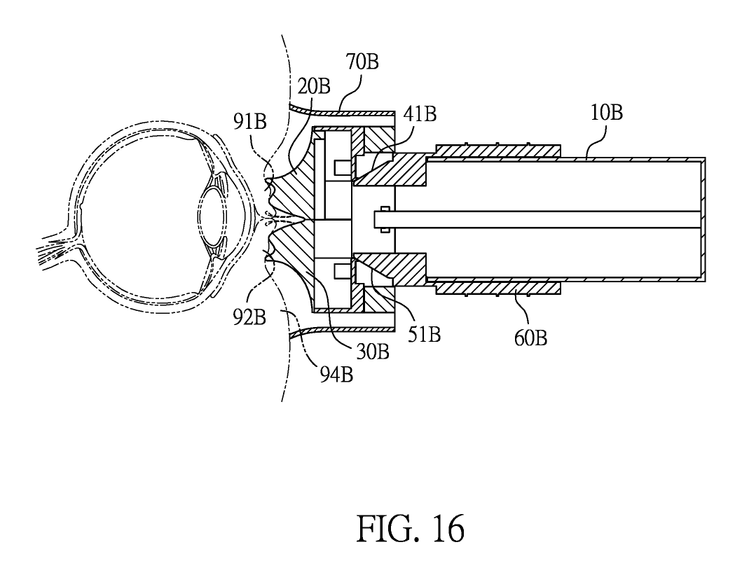

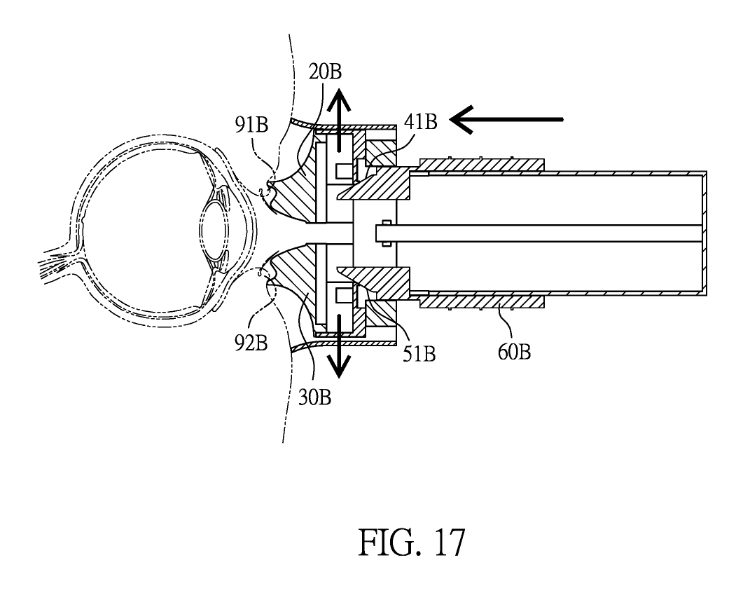

With reference to FIG. 16 and FIG. 17, when pushing the pushing sleeve 60B relative the housing 10B, the pushing sleeve 60B pushes the upper eyelid pusher 20B upwardly via the first inclined surface 41B, and the pushing sleeve 60B also pushes the lower eyelid pusher 30B downwardly via the second inclined surface 51B. By the pushing from the pushing sleeve 60B, the structure of the second embodiment can also push the user's upper eyelid 91B and the lower eyelid 92B apart from each other and hold the eyelids open.

Besides, in the second embodiment, the eye positioning unit is an eye socket positioning sleeve 70B, which means the eye socket positioning sleeve 70B is positioning through the contact with the eye socket 94B. The eye socket positioning sleeve 70B is connected to the housing 10B and is mounted around the upper eyelid pusher 20B and the lower eyelid pusher 30B. An end of the eye socket positioning sleeve 70B is used to abut against the user's eye socket 94B.

In a preferred embodiment, the eye socket positioning sleeve 70B may be an independent component detachably mounted on the housing 10B. Therefore, the eye socket positioning sleeve 70B may be replaced to keep hygienic when used for different users.

When using the second embodiment, the first act is to position the eyelid opener through the eye socket positioning sleeve 70B. After the upper eyelid pusher 20B and the lower eyelid pusher 30B are respectively abutted against the upper eyelid 91B and the lower eyelid 92B, push the pushing sleeve 60B against the housing 10B so as to upwardly move the upper eyelid pusher 20B and downwardly move the lower eyelid pusher 30B. As a result, the upper eyelid 91B and the lower eyelid 92B will be separated and held open.

Because the pushing direction of the pushing sleeve 60B and the direction the for the eye socket positioning sleeve 70B are both toward the user's face, after positioning the eye socket positioning sleeve 70B against the face, the pushing sleeve 60B can be pushed conveniently, and therefore the second embodiment is easier to use compared to the first embodiment, meanwhile encompassing all the advantages derived from the first embodiment.

The eye socket positioning sleeve 70B can also shield the sight of the user, offering a stabilized sensing condition.

In the two different embodiments as shown above, by positioning the eyelid opener through abutting against the side of the nasal bridge or abutting against the eye socket, the eye positioning units are the nasal bridge abutting base and the eye socket positioning sleeve respectively, but it is not limited thereto, as long as the eye positioning unit can contact any other part of the face that is near the eye socket to achieve the positioning function.

Furthermore, the structures of the upper pushing mechanism and the lower pushing mechanism are not limited to the structure described in the first embodiment and the second embodiment. For example, the upper pushing mechanism may make the upper eyelid pusher to push the upper eyelid upwardly by means other than pressing and pushing.

Moreover, the figures of the two embodiments shown above both shows the eyelid opening process starts from opening the fully-closed upper and lower eyelids, but it may be designed to start the process from holding opening the eyelids that are slightly open.

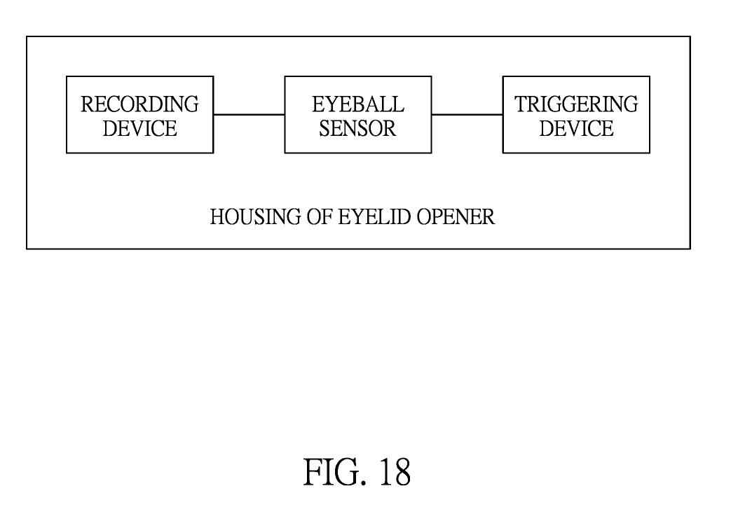

In addition, a button may be added in any one of the two embodiments. By pressing the button the eyeball sensor will be triggered and start the sensing process. With reference to FIG. 18, the eyeball sensor may also be triggered by a triggering device. Specifically, the triggering device is electrically connected to the eyeball sensor. The triggering device may trigger the eyeball sensor according to specific conditions. In the present embodiment, the specific conditions include the results of light sensing, infrared sensing or pressure sensing. The specific condition can also be triggered by mechanical linkages. Several examples are shown as follows.

In the first example, the triggering device is connected to the eyelid opener. When the upper eyelid 91 and the lower eyelid 92 are opened to a certain degree by the eyelid opener, the triggering device will activate the eyeball sensor to start the sensing process. Therefore the sensing process may be automatically activated during the operating of the eyelid opener. Besides, the triggering device may also be a mechanism which is physically activated by the eyelid opener by mechanical linkages. For example, it may be designed to be activated when the upper eyelid pusher 20 or the lower eyelid pusher 30 moves to a specific position. The triggering device may also be designed to be activated by pressure sensing. For instance, the moving of the upper eyelid pusher 20 or the lower eyelid pusher 30 may change the pressure measured by the triggering device. In addition, the triggering device may also be activated by electronic signals. For example, the electronic signals may be transmitted by a sensor further mounted on the eyelid opener, which is capable of detecting the movement or the position of the upper eyelid pusher 20 or the lower eyelid pusher 30.

In the second example, by automatically sensing the changes of the surrounding environment, the triggering device may selectively trigger the eyeball sensor to start the measurement of the eyeball according to specific environmental parameters. For instance, if the setting of the triggering device is to detect the changes of the light, when the user gradually moves the eyelid opener toward the user's eye socket, the housing 10 or the pushing sleeve 60B may shield and darken the light. After detecting the change in the light, the triggering device will then trigger the eyeball sensor to start the sensing process. Moreover, the light detecting function may work in cooperation with the eyelid opener. Specifically, when the upper eyelid 91 and the lower eyelid 92 are opened to a certain degree, the eyeball sensor will first measure the changes in the surrounding environment. When the changes of the surrounding environment meets the conditions set in the triggering device, the eyeball sensor then starts the sensing process of the physiological parameters of the eyeball. Besides, in the present embodiment, the automatic sensing of the surrounding environment may be a built-in function of the eyeball sensor rather than an independent component.

Besides, a recording device may be added to the two examples as shown above, and the recording device is electrically connected to the eyeball sensor and the triggering device. The recording device is capable of recording the sensing results of the eyeball sensor. When the specific conditions for the triggering device to trigger the eyeball sensor is met but continues less than a specific time duration, 4 to 6 seconds in the present embodiment, preferably about 5 seconds, then the recording device stops recording the results of that time. For example, the eyeball sensor may not extract enough data for the physiological parameters of the eyeball if it is sensing for less than 5 seconds, then the recording device can be set to stop recording and not saving the data if the time duration of the darkening of the light is less than 5 seconds, or if the upper eyelid 91 and the lower eyelid 92 are opened for less than 5 seconds.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An eyelid opener for holding a user's upper eyelid and lower eyelid open, and the eyelid opener comprising:
a housing having
an inner space; and
an opening communicating with the inner space;
an upper eyelid pusher disposed in the housing, protruding out of the opening of the housing, and being for pushing the user's upper eyelid;
a lower eyelid pusher disposed in the housing, protruding out of the opening of the housing, and being for pushing the user's lower eyelid;
an upper pushing mechanism connected to the upper eyelid pusher and being capable of moving the upper eyelid pusher upwardly to push the user's upper eyelid upwardly;
a lower pushing mechanism connected to the lower eyelid pusher and being capable of making the lower eyelid pusher push the user's lower eyelid downwardly; and
an eye positioning unit connected to the housing, and being for annularly contacting a surrounding of the user's eye socket.

2. The eyelid opener as claimed in claim 1, wherein the upper pushing mechanism has
a main body mounted in the housing, being pivotable up and down, and having
a connecting end extending toward the opening of the housing and connected to the upper eyelid pusher;
an operating end protruding from the housing and being pressable to operate by the user; wherein the connecting end and the operating end are two opposite ends of the main body respectively;
a first resilient element connected to the main body and the housing, and pivoting the main body in a pivoting direction opposite to a direction in which the upper eyelid is pushed.

3. The eyelid opener as claimed in claim 2, wherein the upper pushing mechanism further has
a second resilient element mounted on the connecting end of the main body and pushing the upper eyelid pusher toward the opening of the housing.

4. The eyelid opener as claimed in claim 1, wherein the lower pushing mechanism has
a lower rod extending obliquely downwardly toward the opening of the housing, and connected to the lower eyelid pusher.

5. The eyelid opener as claimed in claim 4, wherein the lower pushing mechanism further has
a third resilient element mounted on the lower rod and pushing the lower eyelid pusher toward the opening of the housing.

6. The eyelid opener as claimed in claim 1, wherein the lower pushing mechanism is made of an elastic material, and the lower eyelid pusher is connected to an end of the lower pushing mechanism.

7. The eyelid opener as claimed in claim 1, wherein the lower eyelid pusher protrudes farther from the opening of the housing than the upper eyelid pusher.

8. The eyelid opener as claimed in claim 1 further comprising:
a pushing sleeve mounted around the housing, being capable of moving toward the opening of the housing, and selectively pushing both the upper pushing mechanism and the lower pushing mechanism, thereby making the upper eyelid pusher push the upper eyelid upwardly and the lower eyelid pusher push the lower eyelid downwardly.

9. The eyelid opener as claimed in claim 8, wherein:
the upper pushing mechanism is mounted securely on the pushing sleeve and has
a first inclined surface facing upwardly and selectively abutting against the upper eyelid pusher;
the lower pushing mechanism is mounted securely on the pushing sleeve and has
a second inclined surface facing downwardly and selectively abutting against the lower eyelid pusher.

10. The eyelid opener as claimed in claim 1, wherein:
the eyelid opener further comprises
a nasal bridge abutting base mounted on a side of the housing;
the eye positioning unit is a nasal bridge positioning unit mounted on the nasal bridge abutting base, extends toward the opening of the housing, and being for abutting against a side of the user's nasal bridge.

11. The eyelid opener as claimed in claim 10, wherein
the nasal bridge positioning unit protrudes from the opening of the housing farther than the lower eyelid pusher;
the nasal bridge positioning unit protrudes from the opening of the housing farther than the upper eyelid pusher.

12. The eyelid opener as claimed in claim 10 further comprising:
a fourth resilient element mounted on the nasal bridge abutting base, and pushing the nasal bridge positioning unit toward the opening of the housing.

13. The eyelid opener as claimed in claim 10, wherein the nasal bridge positioning unit comprises
an assembling base connected to the nasal bridge abutting base;
an adjusting unit, an end of the adjusting unit detachably mounted on the assembling base;
an abutting part mounted on another end of the adjusting unit, and being for abutting against the side of the user's nasal bridge.

14. The eyelid opener as claimed in claim 10 further comprising:
a nasal bridge track mounted on an outer surface of the housing, and annularly extending to two horizontally opposite sides of the housing;
wherein, the nasal bridge abutting base is mounted on the nasal bridge track and is capable of moving along the nasal bridge track toward the two horizontally opposite sides of the housing respectively.

15. The eyelid opener as claimed in claim 1, wherein the eye positioning unit is an eye socket positioning sleeve connected to the housing and mounted around both the upper eyelid pusher and the lower eyelid pusher; an edge of one end opening of the eye socket positioning sleeve being for abutting against the user's eye socket.

16. The eyelid opener as claimed in claim 1, wherein a light shielding sleeve is formed by a surrounding surface of the housing which is adjacent to the opening of the housing.

17. An eyelid opening and sensing device comprising:
the eyelid opener as claimed in claim 1;
an eyeball sensor disposed in the eyelid opener, and being for sensing physiological parameters of an eyeball via the opening of the housing of the eyelid opener.

18. The eyelid opening and sensing device as claimed in claim 17 further comprising:
a triggering device electrically connected to the eyeball sensor, and being capable of triggering the eyeball sensor to start sensing the eyeball according to specific conditions.

19. The eyelid opening and sensing device as claimed in claim 18, wherein the specific conditions of the triggering device include light sensing, infrared sensing, pressure sensing or mechanical linkages.

20. The eyelid opening and sensing device as claimed in claim 18 further comprising:
a recording device electrically connected to the eyeball sensor, and being capable of recording a result of a sensing process of the eyeball sensor; wherein when a time duration of the specific conditions for the triggering device to trigger the eyeball sensor is less than a specific value, the recording device is stopped by the triggering device.
